(19) United States
(12) Patent Application Publication
(10) Pub. No.: US 2014/0005658 A1
(43) Pub. Date: Jan. 2, 2014

(54) METHOD AND APPARATUS FOR COSMETIC SKIN TREATMENT

(71) Applicant: Avner Rosenbegr, Bet Shearim (IL)

(72) Inventor: Avner Rosenbegr, Bet Shearim (IL)

(73) Assignee: SYNERON MEDICAL LTD., Yoqneam IIIit (IL)

(21) Appl. No.: 13/984,592

(22) PCT Filed: Jan. 26, 2012
(86) PCT No.: PCT/IL2012/000042

§ 371 Date: Sep. 20, 2013

Related U.S. Application Data
(60) Provisional application No. 61/442,361, filed on Feb. 14, 2011.

(51) Int. Cl. A61B 18/18

(52) U.S. Cl. CPC A61B 18/18
USPC 606/33

(57) ABSTRACT

Provided is an apparatus for cosmetic RF skin treatment where the RF energy supplied to the treated skin segment varies in course of the RF energy application period as a function of treated skin segment condition.

### TECHNOLOGY FIELD

The method and apparatus generally relate to skin treatment procedures and in particular to cosmetic skin resurfacing and rejuvenation procedures.

### BACKGROUND

Conventional skin resurfacing or rejuvenation is a known cosmetic skin treatment procedure. Fractional skin resurfacing or rejuvenation is a recently developed skin ablative technology. There are two types of devices used to ablate and heat the skin: laser based devices and RF based devices. Both types of these devices ablate or heat a pattern of extremely small diameter shallow holes or volumes. The holes are microscopically small treatment zones surrounded by untreated skin areas. The treatment results in a very rapid healing or recovery and skin resurfacing of the treated skin segment. In the healing process of the treated zones, a layer of new skin appears, restoring a fresh, youthful complexion.

The pattern of small holes is typically produced by an X-Y scanning laser beam or by application of RF energy to the skin. The laser is focused on the skin and usually operates in pulse mode ablating micron size holes in the skin.

RF based fractional skin treatment produces in the skin a similar to laser pattern of micron size holes. Typically, the energy is delivered to the skin by an applicator equipped by a tip having a plurality of voltage to skin applying/delivering elements or contact elements arranged in a matrix or in an array. The voltage to skin applying elements are placed in contact with the segment of the skin to be treated and driven by a source of suitable RF power and frequency. Application of a high voltage or high power RF pulse to the electrodes ablates the skin under the respective electrode forming a small hole.

Fractional skin treatment is applicable in the correction of almost all cosmetic skin defects such as signs of aging, wrinkles, discolorations, acne scars, tatoo removal, and other skin defects. The cost of the RF based products is lower than that of the products operating with laser radiation and they will most probably become widely used if the treatments requiring control of skin surface ablation and the degree of heat penetration deeper into the skin will be enabled.

### GLOSSARY

In the context of the present disclosure “skin admittance” means the ratio of current phasor to voltage phasor, and “skin impedance” is the inverse of the skin admittance. These complex admittance or impedance can be represented in various ways as a two components real numbers, for example, resistance and phase angle.

“Skin resistance” is the real part of the “skin impedance” or simply ‘impedance”. Both impedance and admittance will be used in the text to describe the skin response to the delivered RF power.

A “phasor” is a complex number that represents both the magnitude and phase angle of a sine electric signal.

The term “skin conductivity” or “electrical skin conductivity” is the reciprocal of “electrical skin resistance” or simply “skin resistance”.

The term “RF energy” has its conventional meaning which is a multiple of RF power by the period of time the RF power was applied or delivered to the treated skin segment.

The term “desired skin effect” as used in the present disclosure means a result of RF power to skin application. The desired skin effect could be wrinkle removal, hair removal, collagen shrinking, skin rejuvenation, and other cosmetic skin treatments.

The term “saline solution” or “saline water” is a term commonly-used for a solution of NaCl in water, more commonly known as salt, in water.

The terms “RF voltage” and “RF power” are closely related terms, the mathematical relationship between these two RF parameters is well known and knowledge of one of them and the load (skin) impedance allows easy determination of the other at a given skin impedance at a certain time, one can control the power delivered to the skin by controlling the voltage of the RF generator. Therefore in practical systems power control is implemented by voltage control.

### BRIEF SUMMARY

An apparatus for cosmetic RF skin treatment by application of the RF energy to the treated skin segment. The apparatus includes an applicator with a tip that is populated by a plurality of voltage to skin applying elements or electrodes located on the tip surface and organized in a number of common clusters. The apparatus applies RF voltage to the electrodes with a magnitude sufficient to cause a desired skin effect. The apparatus continuously or at a high sampling rate senses the treated skin segment electric impedance and dynamically varies in course of an RF power pulse application the RF power delivered and coupled to the skin by changing the driving voltage.

The Dynamic Power Control (DPC) facilitates achieving optimal skin treatment results by adaptation of the RF power into skin introduction to treated skin conditions such as skin resistance, fluid content, and others.

### BRIEF DESCRIPTION OF THE FIGURES

FIG. 1 is a schematic illustrations of a prior art RF apparatus for fractional skin treatment.

FIGS. 2A -2C are schematic illustrations of RF applicator tips for fractional skin treatment according to some examples.

FIG. 3 is a schematic illustration of an RF voltage supplying circuits suitable for driving the present RF applicator tip for fractional skin treatment according to an example.

FIG. 4 is a schematic illustration of an RF applicator for fractional skin treatment according to an example.

FIG. 5 is an exemplary illustration of resistivity of NaCl solution in water as function of solution temperature.

FIG. 6 is an illustration of skin resistivity changes in course of RF voltage pulse application as a function of time for wet and dry skin.

FIG. 7 is a schematic illustration of an RF apparatus for fractional skin treatment according to an example.

FIG. 8 is a schematic illustration according to an example of an RF voltage supplying circuits for driving the RF applicator tip for fractional skin treatment.

FIG. 9 is a schematic illustration of a voltage and current sensing signals mechanism of the apparatus for fractional skin treatment according to an example.

FIG. 10 is a flowchart illustrating a fractional skin treatment method and a controller operating sequence according to an example.

### DESCRIPTION OF EXEMPLARY EMBODIMENTS

The principles and execution of the method and the apparatus may be better understood with reference to the drawings and the accompanying description of the non-limiting, exemplary embodiments, shown in the Figures.

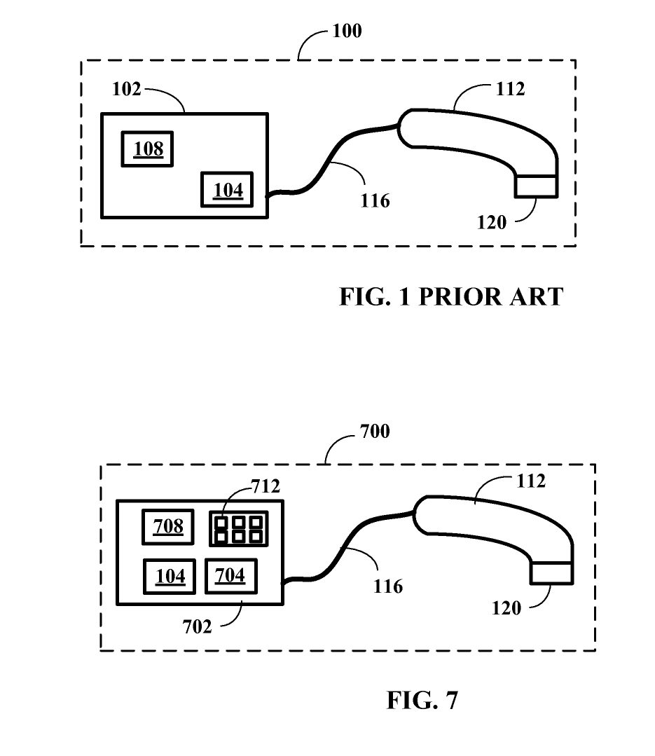

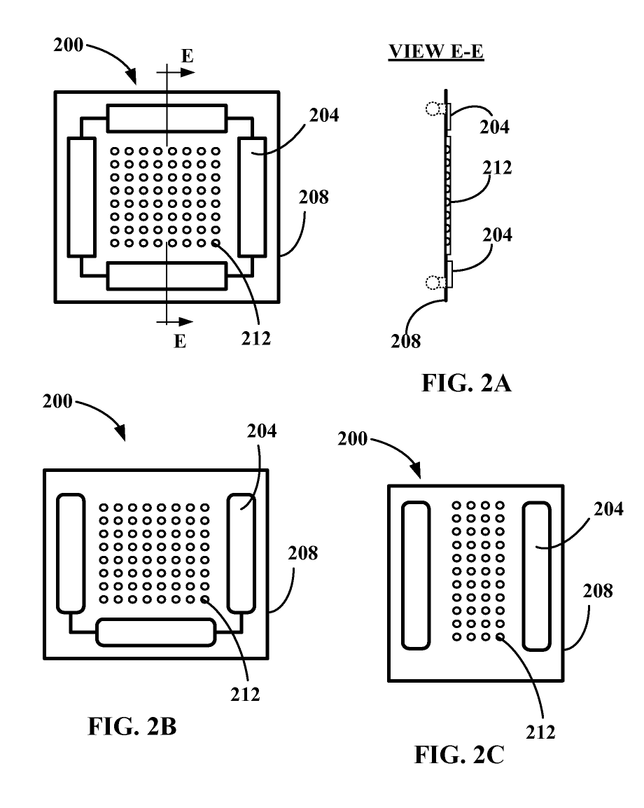

Reference is made to FIG. 1, which is a schematic illustration of an existing apparatus for fractional skin treatment for example, such apparatus as eMatrix commercially available from the assignee of the present application. Apparatus 100 includes an RF voltage supply or generator 104, a controller 108, and an applicator 112. Both RF generator 104 and controller 108 may be located in the same housing 102, although they may be electrically and electromagnetically isolated to avoid electromagnetic interference between them. An umbilical cable 116 connects between applicator 112 and RF power supply or generator 104. Applicator 112 is terminated by a tip 120 that in course of operation is applied to a treated skin segment and delivers RF voltage/power in pulse or continuous mode to that skin segment. Applicator tip 120 may be identical or similar to the tips shown in FIG. 2, although other types of tips could be used. Umbilical cable 116 may conduct the voltage/power supplied by the RF generator to the applicator. Cable 116 may be configured to include cooling fluid tubing and other tubes of wires that may be necessary to fulfill additional functions that could be of use in course of the treatment.

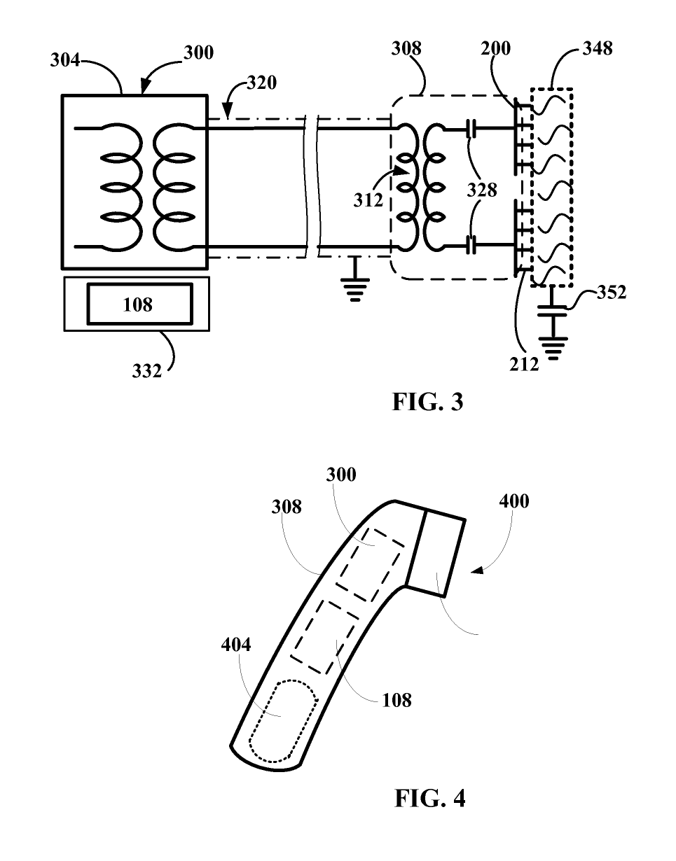

FIGS. 2A-2C are schematic illustrations of RF applicator tips for fractional skin treatment according to some examples. Although the tip 200 is illustrated as a tip for a bi-polar treatment, it may be used for unipolar treatment also. Tip 200 has a first group or cluster of one or more large “ground” electrodes 204 located in the peripheral area of substrate 208 and connected to one or first RF output port of RF generator 104. The second group of electrodes is a cluster of miniature discrete, voltage to skin application elements 212. Voltage to skin application elements 212 arc connected to the other or second port of the RF output transformer (FIG. 3). This particular output port of the transformer may further be configured to have a plurality of output connections such as to enable at least one different parameter of RF voltage supply to each individual voltage to skin application elements or electrodes 212. A particular tip could have 64 elements, although other designs with different number of elements, for example 16, 40, 44, 64, or 144 are possible. The area of the first group of voltage to skin application elements 204 may be substantially larger than the area of the second group of voltage to skin application elements or electrodes 212. The miniature electrodes may have a flat (pancake), needle or dome type shape, of diameter between 100 microns to 600 microns, or between 100 microns to 300 microns. The clusters of the electrodes and more specifically the miniature electrodes may be divided into sub-clusters, including sub-clusters with one electrode only, and each sub-cluster, including an individual electrode, may be driven by RF independent of the others and/or they can be operated sequentially, one after the other, or/and they can be operated concurrently.

FIG. 3 is a schematic illustration of an RF voltage supplying circuits suitable for driving the present RF applicator tip for fractional skin treatment according to an example. The RF voltage supplying circuits are part of the RF generator 104 for driving the present RF applicator tip for fractional skin treatment. An RF voltage generator 300 that includes the RF voltage supplying circuits could be located in standalone housing 304. Alternatively, the RF voltage generator (shown in broken lines) could be located in the applicator case 308. The generator provides RF voltage to applicator tip 200 (FIG. 2), and in particular to voltage to skin delivering elements 204 and 212. The RF voltage is provided through a shielded harness 320 and decoupling transformer 312. Additional series capacitors 328 could be connected between transformer 312 and tip electrodes or voltage to skin delivering elements 212 to filter low frequency components which under certain circumstances could cause unpleasant sensation to a treated subject 348. The length of the harness 320 is selected to facilitate convenient caregiver operation and may be one to two meters long, for example.

Typical operating parameters of the RF generator are: Frequency of the RF: 1 MHz, although any other frequencies between 100 kHz up to 10 MHz may be considered.

Controller 108 (FIG. 1) could govern operation of all of the apparatus. The controller could be located in the same housing 304, although as noted above the controller could be electrically and electromagnetically isolated to avoid electromagnetic interference between the controller and other apparatus elements located in housing 304, or it may be located in a separate housing 332. Controller 108 may have a processor, a memory, and other devices necessary for controlling the treatment process. Controller 108, among others, is operative to set a fraction for RF energy to be delivered into a skin ablative process and a fraction for RF energy to be delivered into a skin non-ablative process. The treated subject is schematically shown by numeral 348. For skin treatment, tip 200 is placed in contact with a segment of the skin segment 350 to be treated and RF voltage is applied to it. The RF induced current passes through the subject 348 and may cause a desired skin effect.

In some examples, as shown in FIG. 4, rechargeable batteries 404, RF voltage generator 300, and controller 108 may be incorporated in the applicator case 308 making the applicator a handheld unit and the use of the applicator 400 independent from a power supply.

Clinical and physical research teaches that there are few parameters which control the amount of skin ablation and internal skin heating. When the apparatus operates with fixed RF power or with fixed RF voltage, the skin properties and specifically skin wetness or humidity play a role in the skin treatment process. Skin properties vary from person to person and even from one segment of skin to the other segment of skin of the same person. Skin properties are affected by environmental conditions such as temperature and humidity, by the various materials applied to the skin before treatment and the by process of skin before treatment cleaning.

The authors of the present disclosure have experimentally found that when a pulse of RF power is applied to the skin the skin impedance is changing in course of the time the pulse is applied to the skin. The authors have proved that the changes or variations in the skin impedance during the time of RF power pulse application can be attributed to the physical processes in the skin. More specifically, the skin properties and their development during the application of the power are manifested in the real and imaginary parts of the electrical impedance.

RF power is delivered by application RF voltage over the skin impedance. The real power delivered to the tissue which causes tissue heating is related to the real part of the skin impedance—the resistance. The imaginary part of the impedance is related to the “reactive power” which delivers no energy to the tissue. At the beginning of the application of the RF voltage to the skin, the upper skin layer—the stratum corneum, may be a poor electrical conductor. Under these conditions the measured current has a very small real part and a large imaginary part, with a phase angle cp (phi) between the current and the voltage close to 90 degrees (current leading). This is probably due to the capacitive nature of this thin upper skin insulating layer (stratum corneum). Skin admittance is the ratio of this current phasor to the voltage phasor, and skin impedance is the inverse of this admittance. Since the real delivered power is (½)V*×I=(½)V*×Y=|V|×|I|×cosφ, almost no energy is delivered to the skin. (In the equation: V—voltage phasor, I—current phasor, Y—admittance, and V*—complex conjugate of V). The treatment is ineffective without power delivery to the skin. To deliver sufficient power the voltage could to be high enough to induce an electrical breakdown of this skin layer.

The authors of the disclosure have found experimentally that at a frequency of 1 MHz, typical threshold voltage for skin breakdown is about 300V (RMS value), and it takes a typical time of 1-5 millisecond to turn this layer into a good conductor and enable power delivery to the tissue or deeper skin layers located beneath this outer skin layer.

According to one aspect of the disclosure, in operation, the system delivers RF voltage to the skin and continuously measures and records the complex (phasor) current, calculates the admittance and/or the resistance, the phase angle between the RF voltage and current and the delivered power. If the phase angle is small (for example, less than 30 degrees or less than 45 degrees) then it is possible to conclude that the upper skin layer is conductive and real power can be indeed delivered to the skin. However, if the phase angle is larger than this value, the system continues to deliver the voltage to the skin for a certain period (1-2 milliseconds), and if the phase angle is not reduced then the controller can increase the voltage and apply it for the next period of time. This process will be repeated until the upper skin layer becomes conductive enough to deliver real RF power to the tissue. This usually happens, when the phase angle between current and voltage is small or the imaginary part of the admittance is equivalently small. Once this target was achieved, the voltage may be increased or decreased to provide the required treatment effect as described below.

It was further found experimentally that the conductive channel created in the skin by this electrical breakdown process is effective for at least a few hundreds of milliseconds even if the delivery of voltage is stopped immediately after the breakdown. The practical implications of this finding mean that after the initial skin (stratum corneum) breakdown has taken place, it is possible to reduce the treatment voltage. For example, to reduce the level of skin ablation, and/or use multiple pulses with delay between them, without loss of the conducting path generated at the stratum corneum layer.

Under wet or humid skin conditions the external layer of skin is conductive from the beginning of the application of the RF voltage, the system will immediately detect current and voltage almost in phase (negligible imaginary part of admittance or impedance), and the treatment can continue to get the desired skin effect as described below.

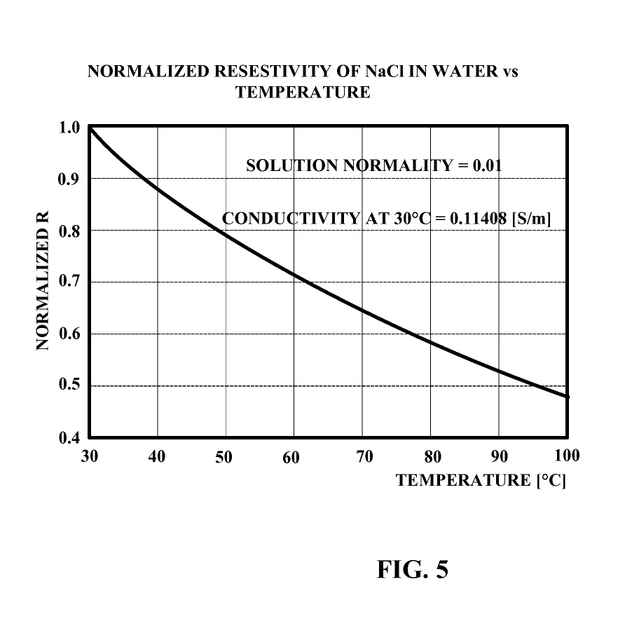

The process of skin heating without ablation is characterized by decrease or drop of skin resistance (real part of the impedance) following from decrease of skin resistivity as the RF voltage is delivering power to the skin. The decrease in skin resistance is most probably related to the basic temperature dependence of the resistance of saline water, since human body consists of about 55%-75% of saline water or solution. FIG. 5 is an exemplary illustration of resistivity of NaCl solution in water as function of solution temperature. It can be seen that from normal skin temperature of about 30 degrees Celsius and up to boiling point of 100 degrees Celsius (which may be considered approximately as the start point of ablation) the resistivity drops to about one third of its initial value.

The RF power could be applied in a pulse mode. The pulses could have different amplitude and duration facilitating achievement of the desired skin effect. When the skin is wet or humid, skin resistance is low, and it drops further with the delivery of RF power into the tissue. Under such conditions and because of heat loss by conduction and convection to surrounding tissue, the applied RF power may not reach the skin ablation phase. Experimentally it was found (and also modeled theoretically), that the temperature of the tissue could maintain about a constant value below boiling point (about 100 degrees Celsius) due to a stable equilibrium between RF power delivery and power loss by heat conduction and convection. Under these conditions the tissue will not be ablated. U.S. patent application Ser. No. 12/505,576 to the same assignee teaches that by increasing the time of the pulse, and thereby increasing the RF energy delivered to the skin, the skin can be driven into the ablative phase. The physical explanation is that as the time increases tissue is further heated and the heat loss decreases, so the delivered RF power can overcome the losses and drive the tissue to ablation. The drawback of this method is that in some cases the surrounding tissue is heated too much, and may cause skin burns. To solve this problem, according to the present method the RF voltage is increased dynamically during the pulse duration, thereby increasing the delivered power, until ablation is detected by the impedance variations.

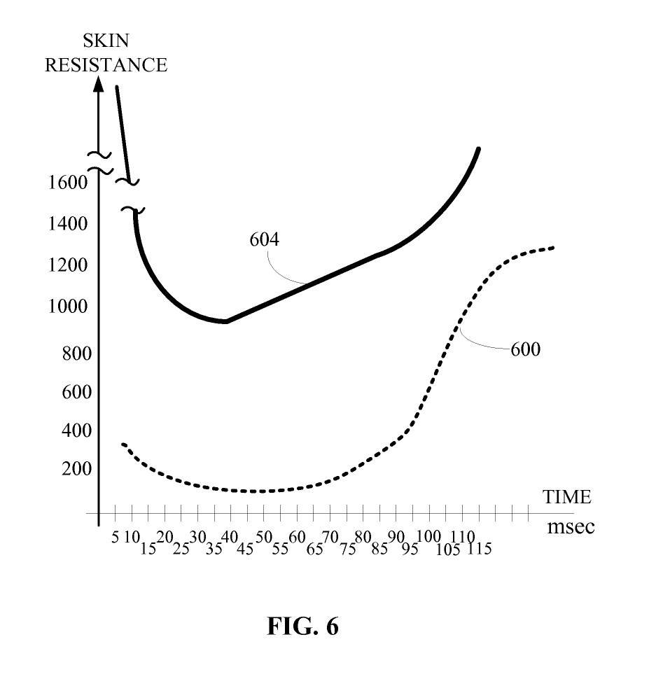

FIG. 6 is an illustration of skin resistivity changes in course of RF voltage pulse application as a function of time for wet and dry skin with constant power vs. resistance curve of the RF power source (generator). Numeral 600 marks wet or humid skin resistance variations. As it can be seen, with wet skin the resistance drops during the first 30 milliseconds due to the process described above, then it maintains for a certain time an almost constant resistance, a manifestation of the equilibrium between RF power delivery and heat loss. Then, if the RF power delivery continues, the surrounding tissue becomes also heated. The skin temperature begins to increase, the boiling point is reached, ablation starts, and this process is manifested as a fast increase in resistance.

When the treated skin segment is dry, RF voltage application is characterized by initial high, as compared to the wet skin, skin resistance and as described above, by a significant capacitive part of the impedance, until the upper stratum corneum is electrically broken. Typically, if the applied voltage is above skin electrical breakdown threshold it takes few milliseconds to turn the stratum corneum into a current conducting state. However in most cases with dry skin, after this initial skin breakdown, the resistance is typically higher than that with wet skin. Sometimes it decreases slightly, or persists at that level for some time then it typically rises slowly during the application of RF energy (numeral 604 in FIG. 6). It is believed that this is because the dry external skin layer begins to ablate almost immediately after the small amount of water contained in it is vaporized. Typically, the treatment is performed by pulses of 10-500 msec duration. The end result of such a pulse to skin application is high ablation but lower heating of internal tissue as compared to pulses applied to the wet skin. There are also significant differences in patient pain level associated with the treatment between the different pulses and their application to different skin conditions.

The variety of skin types and skin conditions and associated with them variations in skin treatment procedures complicates almost every skin treatment as well as achievement of a desired skin effect or treatment result. The current disclosure suggests introduction of a dynamic control of coupled and delivered to the treated skin RF power. FIG. 7 is a schematic illustration of an RF apparatus for fractional skin treatment according to an example. In order to implement the Dynamic Power Control (DPC) a skin treatment apparatus 700 includes a mechanism 704 operative to continuously measure or monitor the electrical impedance (The current disclosure measures skin impedance and extracts or derives from the measurement impedance components such as resistance/capacitance and phase angle.) of the treated skin segment during the RF voltage or RF power pulse application and a control mechanism 708 operative to receive and record the measured impedance, calculate the amount or fraction of energy delivered into the non-ablative process and the amount of energy delivered into the ablative process, and adapt the RF power applied to the treated skin segment condition. Following this a comparison of the fraction of energy delivered into the skin to the respective selected fraction of the RF energy to be delivered may take place. In some embodiments controller 108 (FIG. 1) could include the functions of control mechanism 708. Apparatus 700 could also include a keypad 712 or a touch display with the help of which the caregiver may enter the current treatment parameters. The duration of a typical RF power treatment pulse length is 10-200 msec; therefore the mechanism for measuring of impedance and the mechanism responsible for RF energy to skin conditions adaptation should be fast enough to match these times. The measurement mechanism could operate in continuous mode or may sample the impedance of the treated skin segment every one, or three, or five millisecond, or any other suitable time interval. The control mechanism responsible for RF power to skin conditions adaptation may be operated at a similar time intervals or in a continuous mode.

The operation of apparatus 700 and in particular the RF voltage generator 104 for driving the present RF applicator tip for fractional skin treatment will be explained now. Mechanism 704 continuously measures the electrical impedance of the skin and impedance variations in course of the RF voltage pulse application. In order of getting the most accurate skin impedance sensing (and derive from it skin resistance or/and capacitance and/or phase angle) it is best to measure the current and voltage as close as possible to the treated skin segment. This way the parasitic effects of stray capacitance, cable and transformer loses are avoided.

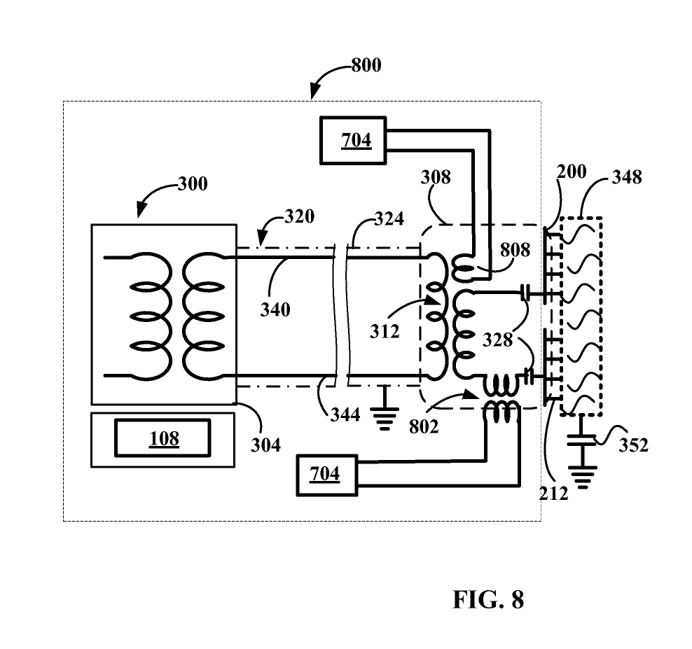

FIG. 8 is a schematic illustration of another example of the RF voltage generator for driving the RF applicator tip for fractional skin treatment. The RF voltage generator 800 for driving the present RF applicator tip 200 includes a current sensor 802 located after the final decoupling transformer 312 and a voltage sensor 808 which is effected by adding one or more windings to the secondary coil of the decoupling transformer 312. The voltage on these windings equals to the output voltage at the secondary coil divided by the ratio of the number of windings of the secondary coil to the number of the sensing winding. The continuously sensed voltage and current signals are communicated to monitoring mechanism 704 operative to monitor and derive from the measurements the electrical impedance and/or derive skin admittance and/or resistance and phase angle of the skin segment during the RF voltage pulse application. In some embodiments controller 108 may include the functions of monitoring mechanism 704 and operate instead mechanism 704.

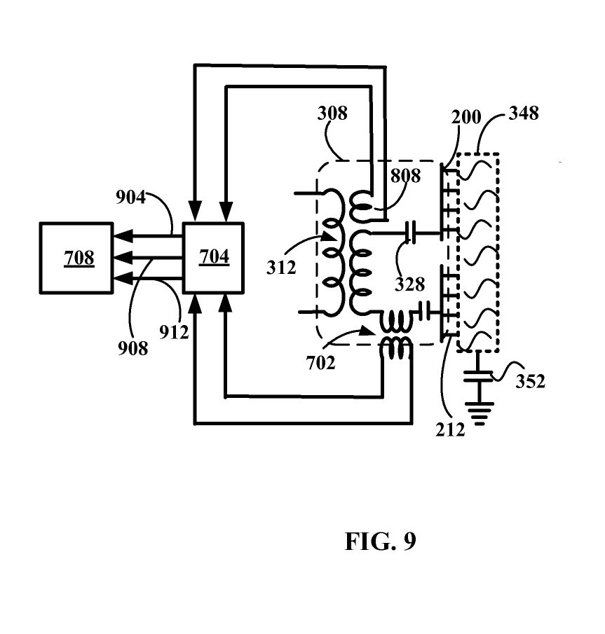

FIG. 9 is a schematic illustration of a voltage and current sensing signals monitoring mechanism of the apparatus for fractional skin treatment according to an example. The electronic circuit of monitoring mechanism 704 could include true RMS processors deriving the absolute values of voltage V and current I and multiplying device which provides the true (real) RF power delivered to the load, which in this case is tip 200 (FIG. 2) attached to the treated skin segment 348 (FIG. 3). In course of apparatus 700 (FIG. 7) operation, the signals sensed by current sensor 802 and voltage sensor 808 are communicated to monitoring mechanism 704 that processes the sensed signals and transforms them by an Analog-to-Digital converter into digital values of true RMS voltage (Vₜᵣᵤₑ) 904, true RMS current (Iₜᵣᵤₑ) 908, and true RF power value (Pₜᵣᵤₑ) 912. Digital values of the processed signals are sent to the control mechanism 708 that based on the absolute value of current, voltage and true power, expressed as |V|×|I|×cos φ, can compute (based on widely available know-how) the phase between current and voltage, the complex admittance/impedance and the real value of the impedance—the resistance.

The caregiver or system operator, or even the user itself with the help of mechanism 704 operative to measure the electrical impedance/resistance/admittance of the skin segment during the RF voltage pulse application, can define the type of the desired treatment and the control mechanism 708 will be set to operate and establish the desired treatment parameters. The parameters may be set to cause skin ablation, skin heating, and a mix of skin ablation and skin heating.

The operation of the sensing and control of the apparatus will be explained now in detail. From the starting point there is a cyclic routine of application, sensing and setting voltage for the next cycle. The first cycle begins by application of a arbitrary voltage which could be a voltage such as 50-1000 volt or more typically 100-500 volt for a certain period of time which may be few hundreds of microseconds to few millisecond. During this period and/or at the end of it, skin impedance (resistance/capacitance/phase) is measured and the treated skin segment conditions are determined. Based on this measurement and as will be explained below accounting for skin resistance and the phase angle (φ) between the RF voltage and current induced by the applied RF voltage the voltage for the next period of time is set by the controller. In the subsequent cycles the controller sets the voltage according to the last and all previous measurements of the skin impedance. The periods may be few millisecond or shorter—the minimum cycle duration is typically determined by the sensors and controller response time, although practically it may be a quasi-continuous sensing and control process.

The use of the voltage as a control parameter is technically convenient since most power supplies are voltage controlled power supplies. Controlling the voltage enables control of the delivered to the skin RF power. Since the impedance is measured and known (and skin resistance/admittance may be calculated), the delivered power is simply the square of the voltage divided by the load, which is skin resistance (real part of the impedance), so for setting a certain level of power one can set the level of voltage which delivers this power to the load. According to another embodiment of the present method, the method may use a power controlled source, and control the RF power. In still a further embodiment it is possible to use current controlled RF source. Although for the purpose of explanation of the method, the controlled RF voltage embodiment will be used, it is to be understood that controlled RF power and controlled current sources can also be used.

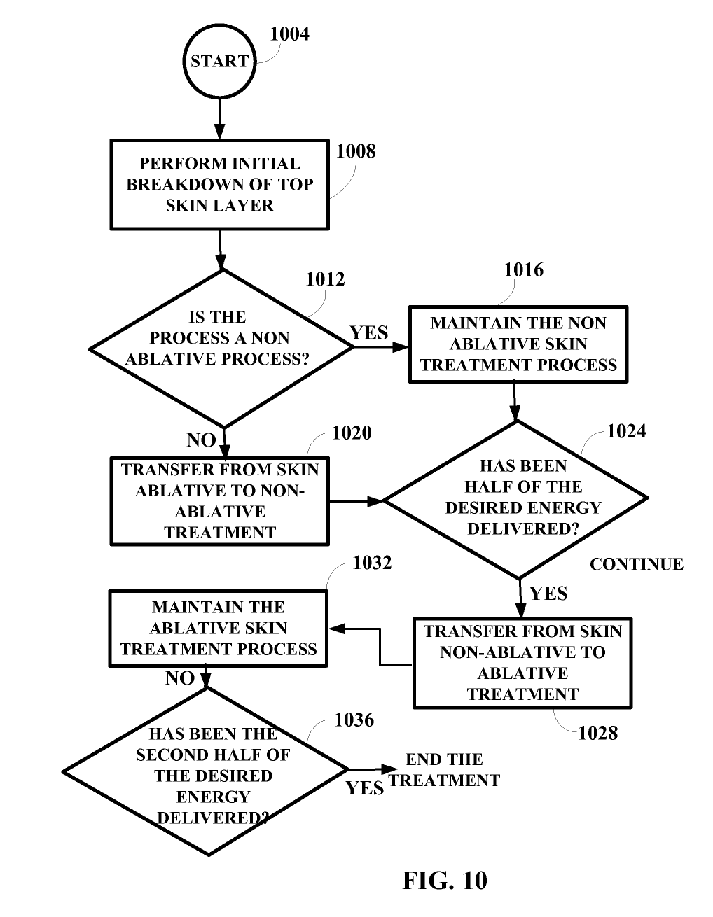

The operation of controller sequence and related with it tasks and processes are described below, and shown schematically in FIG. 10. The caregiver may enter with the help of keypad 712 (FIG. 7) his/her treatment preference, which may include the degree of skin ablation and total amount of delivered to the skin energy (or equivalently energy per pin/contact in the tip). The degree of skin ablation may be set from none skin ablation to a very high degree or level of skin ablation. In intermediate settings the controller may be operative to deliver a certain fraction or amount of the desired energy without causing a non-ablative skin treatment process; the rest of the energy may be delivered to cause a skin ablative process. The fraction or amount of the energy delivered without causing skin ablation and the rest of the energy delivered to cause a skin ablative process may be set by the caregiver with the help of a controller controlling delivery of the energy to the applicator. Accordingly the controller may include the following control functions or processes that enable implementation of treatment tasks such as:
(a) Perform initial electrical break down of the outer skin layer (typically the stratum corneum);
(b) Maintain a skin non-ablative treatment process;
(c) Maintain a skin ablative process at a certain level;
(d) Perform transition from non-ablative to ablative process;
(e) Perform transition from ablative to non-ablative process.

The function or process of performing initial electrical break down of the outer skin layer (Block 1008) is operative in all dry skin treatments. With wet skin there is no need for this breakdown, since it is conductive enough. However, operation of functions or processes marked as (b) through (e) depends on the caregiver setting. In all cases the tasks and processes are based on the measured impedance/admittance/resistance from the beginning of the pulse up to the decision time for the next time period. For example, the decision process may include use of phase angle between current and voltage or equivalently admittance phase angle, the last value of the skin resistance, average values of resistances measured over a certain time, slope of the resistance vs. time at a certain time before the decision making time. The controller, if necessary, may undertake correcting actions which may include the RF voltage increase, if the phase angle and skin resistance are above the preset values, RF voltage decrease, if the phase angle and skin resistance are below the preset values and completely ceasing RF voltage to skin delivery for a certain period of time. From the measured data the controller may derive the amount of energy delivered up to the decision time and may respond by ceasing the RF to skin delivery when the required energy was delivered, or performing non-ablative to ablative transition process (d) when the energy delivered is equal or greater than the fraction of total energy required to be non-ablative in the caregiver setting.

Below are more detailed examples of the processes according to the present method. The task or process (a) of performing initial electrical break down of the outer skin layer (Block 1008) is operative for the first few milliseconds, for example between 0.5 msec to 5 msec. The aim of the process is to make sure that the stratum corneum has been electrically broken down or perforated to enable effective power delivery into the skin and tissue. Therefore a certain voltage V1 is applied for the first period of time (first cycle). The phase angle φ between current and voltage of equivalently admittance phase angle is measured as well as the resistance R. If φ is above a certain preset value φ₁ the controller concludes that the skin is dry and was not broken through. In this case the voltage will be increased to a value V2>V1. This process will be repeated in each time cycle until a skin breakdown is achieved and phase angle φ becomes smaller than φ₁. When φ<φ₁ the voltage is reduced to a lower value V3<V1. This reduction of voltage to V3 is necessary to prevent exaggerated skin ablation, since initial breakdown voltage is high, and if this high voltage is maintained after the breakdown it may deliver a larger than desired power. If initially, the measured phase angle φ is smaller than φ₁, it is indicative of wet skin and no need to affect the skin breakdown. Additionally, the controller may check also the value of the skin resistance R. If the value of R is above a pre-set value the controller can increase the voltage to effect electrical breakdown of the stratum corneum, and if the value is lower than the pre-set value the voltage may be reduced to prevent too much ablation. The controller can combine the measured phase angle and resistance to deduce if the skin was electrically broken or not, and accordingly increase or reduce the RF voltage to effect breakdown or to continue the treatment, The lower is the resistance (R) value the wetter is the skin. Controller 708 may have in memory a table with value of skin resistance (R) with each resistance value corresponding to different degree of skin wetness, and according to this table and the type of treatment selection the operator may set the voltage (and accordingly the RF power and energy) for the next step. For example, if resistance is high and the operator setting is for non-ablative skin treatment the voltage will be reduced. Typical value of φ1 may be 15, 30, or 45 degrees. Typical value of V1 may be 200, 400, or 1000 Volts RMS. The value of resistance (R) depends on tip structure. For a tip with 64 pins, each one having diameter of 100 to 250 microns. The value of R before effecting breakdown may be higher than few KOhms. After the initial breakdown the value of resistance for wet skin may be 100 to 600 Ohms, although depending on the degree of skin wetness it may be 100 to 300 Ohms or from 300 to 600 Ohms. The resistance of dry skin is usually between 600 to 1000 Ohms and very dry skin resistance may be above 1000 Ohms. The average value of skin resistance between wet to dry skin is about 600 Ohms. For a tip with a plurality of voltage to skin delivering elements the skin resistance values may vary from 5 KOhm to 100 KOhm per voltage to skin applying element.

The value of resistance R1 used below usually depends of the specific tip structure. For the tip structure described above it is about 600 Ohms.

The task of maintaining a skin non-ablative treatment process (Block 1016) generally may be used to ensure that the non-ablative skin treatment process takes place. At the first few milliseconds control mechanism 708 based on R (resistance) values makes a decision if the process is already ablative or not (Block 1012). If the skin treatment process is already ablative, then the task of transition from ablative to non-ablative process (e) is operated (Block 1020). The skin treatment process may be transferred from non-ablative to ablative treatment (block 1028), if for example, half of the pulse energy, or any other fraction or percentage of the energy such as 20%, 30% or 80% as it may be set through, controller 708 by the caregiver, has been delivered in course of the non-ablative treatment (Block 1024). A non-ablative process is typically characterized by presence of wet skin. The selection or operation of type of process decision may be based on comparing resistance value R to a certain value R1 which is the boundary between wet and dry skin and on the slope of R vs. time. For R<R1 and for negative R slope (FIG. 6) the process is non-ablative and the process of maintaining a skin non-ablative treatment process (b) becomes operative.

As shown in FIG. 5, delivering power to the skin which contains an amount of saline water generates decrease in resistance R until the skin reaches a temperature of 100 degrees Celsius where the process becomes ablative. The ratio between initial resistance R at 30 degrees Celsius and final resistance R at 100 degrees Celsius is about 3:1. To maintain the non-ablative process the voltage has to be at a level which drives the resistance to not less than a certain fraction of the value of the resistance R at the beginning of the pulse. This fraction may be between 0.4 and 0.8 or between 0.5 and 0.7 of the voltage at the beginning of the pulse. If resistance R falls below this value the RF voltage may be reduced, if it is above this value the voltage will be increased.

Another criteria which may be applied as alternative to or in combination with the fraction criteria is based on the slope of resistance R vs. time (FIG. 6). If the slope, which is typically negative, absolute value, is larger than a certain value, the RF voltage will be reduced, if the slope absolute value is smaller than another or the same certain value the voltage will be increased. The optimal slope value may be selected to be such that at the end of the pulse the value of resistance R will not drop below 0.4 of its initial value.

The task of maintaining a skin ablative process (c) (Block 1032) becomes operative when the caregiver wants to maintain the skin treatment process ablative at a certain level. If the skin treatment process is identified as non-ablative as described above, then the transition from non-ablative to ablative process (d) may become operative (Block 1028). The skin ablative process is characterized by resistance R greater than a certain resistance value R2, which may be equal to R1 or some value above R1. Another characteristic of ablative process is that the slope of the resistance R is slightly positive. It was found that high ablative process is manifested as high resistance R, and may be accompanied with patient discomfort. Therefore, one of the optimal ways of reducing this discomfort is to maintain the level of ablation within certain range, although the range may depend on the caregiver decision. Let the resistance range be between R3 and R4, where R4>R3>=R2. Then if R<R3 the RF voltage will be increased, when R>R4 the RF voltage will be decreased. The amount of increase or decrease of the RF voltage may be a function of the resistance differences R-R3 or R-R4. If in course of maintaining the ablative skin treatment process a second half, or other selected portion or fraction of the desired energy has been delivered (Block 1036) the treatment may be terminated.

Obviously there are other ways for changing RF voltage V as function of skin resistance R. For example, a target value R5 can be set, where R4>R5>R3, and the voltage may be set as some monotonic function of difference between R-R5. For example, the voltage change ΔV=−a(R-R5), where “a” is a constant. R3, R4, R5 resistance values depend on the caregiver skin ablation level setting and on the tip structure. For a tip with 64 pins or contact electrodes with diameter 250 microns each, R3 may be between 600 Ohms and 1000 Ohms, R4 between 1000 Ohms and 2000 Ohms or something like 40 to 64 KOhm per pin or 64 to 130 KOhms per pin depending on the treated skin condition.

The task of transition from non-ablative to ablative process (d) becomes operative when the caregiver requires at least a certain amount of skin ablation to take place. This process is based on increasing the RF voltage until the slope of resistance R becomes positive and/or until the value of resistance R is above a certain resistance value R6. Typically R6=R1 or R6=R2. Once these conditions are obtained sub-process or task (c) has to become operative.

The task of transition from skin ablative to non-ablative process (e) once operated, reduces the voltage until resistance R is below a certain resistance value R7. Typically R7<=R1. If during a certain period of time, typically few milliseconds, R does not drop below R7, then the controller 708 (FIG. 7) may cease completely the RF voltage delivery (V=0) for a certain period, which may be between 5 msec and 250 msec or between 10 msec and 100 msec. During this time the tissue becomes colder due to heat conduction and convection, and it becomes wetter since body fluids are flowing into the ablated skin volume. After this period of time the controller turns ON the RF again, raising slowly the value of RF voltage V, and performing a transition to process (b) which keeps the skin treatment process non-ablative.

As an example, assume a caregiver selection of half ablative process, namely half of the energy, or any other percentage such as 20%, 30% or 80% as it may be set through controller 708 by the caregiver, delivered to the skin will not make ablation, the other half or other percentage will be delivered into ablation process. The condition of the skin is unknown. First an initial electrical break down of the outer skin layer (a) is performed. A typical time for completion of this process is 1 msec for wet skin and 1-5 msec for dry skin. Then if a non-ablative process is identified control is turned over to the task of maintaining a skin non-ablative treatment process (b) until, for example, half of the desired energy is delivered. Typical times may be between 10 msec and 200 msec. Then the task of executing the transition from skin non-ablative to skin ablative process (d) is performed and it is followed immediately by the task of maintaining a skin ablative process at a certain level (c) which is operated until the second half or other selected fraction of the desired energy is delivered.

The following table summarizes the tasks and processes.

| Task and | | |
|---|---|---|
| process | Task | Operational Time |
| (a) | Initial electrical breakdown | Always at the beginning of a |
| | of top skin layer | pulse |
| (b) | Maintain non-ablative | Caregiver selects at least part |
| | process | of the pulse non-ablative |
| (c) | Maintain ablative process at | Caregiver selects at least part |
| | a certain level | of the pulse ablative |
| (d) | Transforming from non- | (1) Caregiver selected at least |
| | ablative to ablative process | part of the pulse ablative and |
| | | pulse started non-ablative |
| | | (2) Required process ablative |
| | | and actual becomes different. |
| (e) | Transforming from ablative | (1) Caregiver selected at least |
| | to non-ablative process | part of the pulse non-ablative |
| | | and pulse started ablative |
| | | (2) Required process non- |
| | | ablative and actual becomes |
| | | different. |

The use of voltage setting is one convenient way to control to power for a certain load (skin) resistance. Another way is to make an RF generator with a control which sets the output power to a specified value for a range of load resistances. The control steps may be chosen so that the variations of R during each step is small, so a good definition of power for each step could be obtained by controlling the voltage and vice versa.

It also possible to summarize the three disclosed methods of cosmetic RF skin treatment exists. In course of one skin treatment processes initially selection of a fraction for RF energy to be delivered into a skin ablative process takes place, than selection of a fraction for RF energy to be delivered into a skin non-ablative process takes place. A certain RF voltage pulse is then applied to a treated skin segment and continuous measuring and recording of the treated skin segment resistance and the delivered RF power is performed. All of the measurements and recording are performed in course of the RF pulse application. A monitoring mechanism 704 is continuously monitoring the recorded RF resistance of the treated skin segment and the recorded RF power to calculate the fraction of energy delivered into the non-ablative process and the fraction of energy delivered into the skin ablative process and it compares the fraction of energy delivered into the skin to the respective selected fraction of the RF energy. Depending on the outcome of the comparison, the RF voltage may be set to a value causing a non-ablative skin treatment process if the fraction of energy delivered into the non-ablative process is smaller than the respective selected fraction of the energy to be delivered in said process. Otherwise the RF voltage may be set to a value causing an ablative skin process until the respective selected fraction of energy set for the ablative skin treatment is obtained.

Another method of cosmetic skin treatment by application of RF energy to the treated skin segment includes applying a certain voltage level to a treated skin segment, determining the skin condition by: a) measuring skin resistance (R); and b) determining phase angle (q) between the RF voltage and current induced by the applied voltage. If the phase angle and skin resistance are above preset values, increasing the RF voltage applied to the treated skin segment to cause an electrical skin breakdown and if the phase angle and skin resistance are below certain preset values, reducing the RF voltage applied to the treated skin segment and continuing the skin treatment.

In still an additional method of cosmetic fractional RF skin treatment a selection of a desired skin treatment process from a group of skin treatment processes consisting of skin ablative and skin non-ablative process is performed. Initially, an RF voltage pulse is applied to a treated skin segment and in course of the RF voltage pulse application measurement and recording of the treated skin segment RF resistance is performed. In course of the RF pulse application the treated skin segment recorded RF resistance is continuously monitored to determine the type of the skin treatment process—ablative or non-ablative. The RF voltage to drive the selected process setting is based on the result of treatment process type. The RF voltage set may be increased if the selected process is ablative and the monitored skin process is a non-ablative process, and the RF voltage could be decreased if the selected process is non-ablative and the monitored skin treatment process is ablative.

The disclosed above method of fractional skin treatment provides a reliable control over the skin treatment process, enables selection between skin ablation and skin heating, reduces RF power to skin application time, and facilitates easier achievement of a desired skin effect. The electric scheme and the tip structure disclosed above also eliminate electrical shock feeling, reduce or eliminate the pain associated with the treatment and increase the treatment efficacy.

1.-45. (canceled)

46. An apparatus for cosmetic RF skin treatment, said apparatus comprising:
a controller operative to set a fraction for RF energy to be delivered into a skin ablative process and a fraction for RF energy to delivered into a skin non-ablative process;
an applicator operative to apply RF energy pulse to a treated skin segment corresponding to the set fraction of RF energy;
a mechanism operative to measure and record the treated skin segment impedance and the delivered RF energy and communicate the values to a mechanism operative in course of the RF pulse application to continuously monitor the recorded impedance of the treated skin segment and the recorded RF energy and to calculate the fraction of energy delivered into the non-ablative process and the fraction of energy delivered into the skin ablative process; and
comparing to the fraction of energy delivered into the skin to the respective selected fraction of the RF energy.

47. The apparatus according to claim 46, wherein said controller controls delivery of RF energy to the skin by:
setting the RF voltage to a value causing a non-ablative skin treatment process in accordance with respective selected fraction of the energy to be delivered in said process; and
setting the RF voltage to a value causing an ablative skin process until the respective selected fraction of energy set for the ablative skin treatment is obtained.

48. The apparatus according to claim 47, further comprising a mechanism operative to derive from the skin impedance skin resistance and to measure and record the treated skin segment resistance and the delivered RF power and communicate the values to a monitoring mechanism operative in course of the RF pulse application to continuously assess if the skin treatment process is an ablative or non-ablative process.

49. The apparatus according to claim 46, wherein the controller performs transition from a non-ablative skin treatment to ablative skin treatment by increasing the applied RF voltage and transition from ablative skin treatment to non-ablative skin treatment is performed by decreasing the applied voltage.

50. The apparatus according to claim 49, wherein the controller setting the applied RF voltage value to a value causing a non-ablative skin treatment process and to a value causing an ablative skin process is a function of skin resistance and phase angle.

51. The apparatus according to claim 47, wherein the controller terminates the ablative skin treatment process when the selected fraction of the RF energy is delivered to the treated skin segment.

52. The apparatus according to claim 46, wherein the selected fractions of RF energy are delivered to the treated skin segment between 10 msec and 200 msec.

53. The apparatus according to claim 46, wherein the applicator includes a tip with a plurality of voltage to skin delivering elements and wherein the skin resistance values vary from 5 KOhm to 100 KOhm per voltage to skin applying element.

54. An apparatus for cosmetic RF skin treatment, said apparatus comprising:
an applicator including a tip with a plurality of RF voltage to skin delivering elements;
an RF voltage source operative to drive the applicator and apply a certain RF voltage pulse to a treated skin segment, said applicator including a current sensor and a voltage sensor operative to sense the voltage applied by the applicator and current induced by said voltage in the treated skin segment and generate corresponding signals;
a mechanism receiving and recording said voltage and current signals and deriving from said signals at least one of a group of a RF resistance of the skin segment, true RF power delivered to the skin segment and phase angle between the RF voltage and induced by said voltage current values and communicating said values to a controller; and
wherein said controller based on the recorded RF resistance of the treated skin segment and the recorded RF power calculates the fraction of energy delivered into the non-ablative process and the fraction of energy delivered into the skin ablative process.

55. The apparatus according to claim 54, wherein said tip includes two types of RF voltage to skin delivering elements and wherein at least one of the types of RF voltage to skin delivering elements is a plurality of miniature electrodes.

56. The apparatus according to claim 54, wherein said RF voltage source is operative to supply RF voltage to said voltage to skin delivering elements has two ports with a plurality of voltage to skin delivering elements connected to a first RF port, and voltage to skin delivering elements bounding the plurality of voltage to skin delivering elements connected to a second RF port.

57. The apparatus according to claim 54, wherein the controller is
setting the RF voltage to a value causing a non-ablative skin treatment process if the fraction of energy delivered into the non-ablative process is smaller than the selected fraction of the energy to be delivered in said process; and
to a value causing an ablative skin process until the selected fraction of energy set for the ablative skin treatment is obtained.

58. The apparatus according to claim 57, wherein for transition from a non-ablative skin treatment to ablative skin treatment the controller increases the applied voltage and for the transition from ablative skin treatment to non-ablative skin treatment the controller decreases the applied voltage.

59. The apparatus according to claim 57, further comprising measuring and recording the phase angle between the voltage and current, monitoring the recorded angle value and including said angle value in assessing if the process is ablative or non-ablative.

60. The apparatus according to claim 57, wherein the setting of RF voltage to a value causing a non-ablative skin treatment process and to a value causing an ablative skin process is a function of skin resistance and phase angle.

61. The apparatus according to claim 60, wherein the controller terminates the ablative skin treatment process when the selected fraction of the RF energy is delivered to the treated skin segment.

62. The apparatus according to claim 57, wherein the selected fractions of RF energy are delivered to the treated skin segment between 10 msec and 200 msec.

63. An apparatus for cosmetic skin treatment by application of RF energy to the treated skin segment, said apparatus comprising:
an applicator operative to applying a certain voltage level to a treated skin segment;
a monitoring mechanism operative to determine skin condition by measuring skin impedance and calculating skin resistance (R) and determining phase angle (φ) between the RF voltage and current induced by said voltage and communicating determined values to a controller; and
wherein said controller increases the RF voltage applied to the treated skin segment to cause an electrical skin breakdown and form in the skin an electrically conductive channel if the phase angle and the skin resistance are above preset values; and
reduces the RF voltage applied to the treated skin segment if the phase angle and skin resistance are below certain preset values; and continues the skin treatment.

64. The apparatus according to claim 63, wherein the certain voltage level is between 50 volt and 1000 volt.

65. The apparatus according to claim 63, wherein the certain voltage level is between 100 and 500 volt.

66. The apparatus according to claim 63, wherein the preset values of the phase angle are less than 45 degrees.

67. The apparatus according to claim 63, wherein the preset values of the phase angle are less than 30 degrees.

68. The apparatus according to claim 63, wherein the cosmetic skin treatment is a fractional cosmetic skin treatment.

69. The apparatus according to claim 68, wherein the fractional cosmetic skin treatment is performed by an applicator including a tip with a plurality of voltage to skin delivering elements and wherein the skin resistance values vary from 5 KOhm to 100 KOhm per voltage to skin applying element.
